(19) United States
(12) Patent Application Publication
(10) Pub. No.: US 2017/0056536 A1
(43) Pub. Date: Mar. 2, 2017

(54) ELECTROCHEMICAL MANIPULATION OF IMPLANTS

(71) Applicant: RUSH UNIVERSITY MEDICAL CENTER, CHICAGO, IL (US)

(72) Inventors: Nadim James Hallab, Oak Park, IL (US); Ryan Freed, Chicago, IL (US); Dmitry Royhman, Chicago, IL (US); Steven P. Mell, Chicago, IL (US)

(73) Assignee: RUSH UNIVERSITY MEDICAL CENTER, CHICAGO, IL (US)

(21) Appl. No.: 15/249,733

(22) Filed: Aug. 29, 2016

Related U.S. Application Data
(60) Provisional application No. 62/213,390, filed on Sep. 2, 2015.

(51) Int. Cl. A61L 2/03; C25F 1/00

(52) U.S. Cl. CPC A61L 2/035; C25F 1/00; A61L 2202/21

(57) ABSTRACT

The present invention generally relates to systems and methods of treating microbial growth on a conducting surface, for example, the conducting surface of an implanted medical device. One embodiment of the method includes applying a electrical current through the conducting surface and a counter electrode. The electrical current is varied such that an electrical potential of the conducting surface alternates compared to the electrical potential of a reference electrode and where the electrical current is varied to an extent sufficient to reduce or prevent microbial growth on the conducting surface.

### RELATED APPLICATIONS

The present patent application claims the benefit of the filing date of U.S. Provisional Patent Application No. 62/213,390, filed Sep. 2, 2015, the contents of which is hereby incorporated by reference.

### TECHNICAL FIELD

The present invention generally relates to systems and methods for treating microbial growth on a conducting surface, for example, the conducting surface of an implanted medical device. One embodiment of the method includes varying the electrical potential of the surface to an extent sufficient to reduce or prevent microbial growth on the surface.

### BACKGROUND

Amputation is the removal of a body extremity by trauma or surgical procedure. (1-4) Examples of trauma include loss of extremities in heavy machinery or car accidents or surgical amputation in cases of cardiovascular disease, diabetes, or cancerous tumors. (1) The remaining limb is commonly referred to as the residual limb. There are currently many strategies to artificially restore the residual limb's functionality. (1,3,5) Osteointgrated replacement implants or limbs are not commonly used due to risk of infection.

Osseointegrated endoprosthesis attachment is a procedure in which an implant is fitted into the medullary canal of the residual limb with a component of the implant protruding from the skin to connect to the external prosthetic limb. (1) Treatment with osseointegrated prostheses have been shown to improve the quality of life; however, infection remains a primary risk. (5) In order to prevent traumatic revision surgery, it is necessary to actively mitigate infection, even years after the surgery.(6) There have been reports of problems with this implant system, including loosening of the implants, implant fractures, deep bone infections, superficial soft-tissue infections, and troublesome discharge. (2,4) The Micro-Shock therapy device provides a convenient, non-invasive treatment of bacterial infection for osseointegrated endoprostheses. (7)

Multidrug resistance of bacteria is becoming a major concern to public health as bacteria are becoming more resistant to drug by both intrinsic and acquired mechanisms. (8,9) Intrinsic mechanisms come from specific cellular mechanisms and structures such as drug extrusion efflux pumps. (9) Furthermore, bacteria can acquire resistance through genetic mutations and horizontal gene transfer.(9)

Bacterial colonize an implant surface and secrete a “biofilm” whereby the bacteria secrete an extracellular matrix, which protects them from the harsh external environment and the immune system.(9) Thus bacteria are readily able to tolerate systemic antibiotics. In addition, bacterial cultures often contain a small fraction of persister cells (dormant cells), which are not metabolically active. (9) This presents a problem because most antibiotics only target metabolically active cells. (9) Persister cells can survive at much higher antibiotic concentrations. (9) The ones that do survive can revert to an active state and regenerate the bacterial population. (9)

By creating a bactericidal charge transfer between bacteria and conductive or semi-conductive implant surface this denies bacteria a surface to fortify themselves against immune attack. For example, a total current of 100 mA DC applied to surgical stainless steel pins has been found to prevent Staphylococcus epidermidis infection around percutaneous pins in goats. (12) Bacteria cell surfaces contain various proteins and biomolecules, which contain electrochemically active groups. (10) As a result the bacterial cell surface contains free electrons, which react with a conductive/active surface. (11)

### SUMMARY OF THE PREFERRED EMBODIMENTS

In one aspect, the present invention provides a method for treating, reducing or preventing microbial growth on a conducting surface. For example, the microbial growth may be associated with an implanted medical device having such a conducting surface. The medical device may be partially or completely implanted within the body of a patient. In one embodiment the method includes applying an electrical current through the conducting surface and a counter electrode. The electrical current is varied such that the electrical potential of the conducting surface alternates compared to the electrical potential of a reference electrode and where the electrical current is varied to an extent sufficient to reduce or prevent microbial growth on the portion of the conducting surface implanted within the body of the patient.

A potentiostat device may be used to vary in the electrical current between the conducting surface and the counter electrode to provide for the varying electrical potential of the conducting surface relative to the reference electrode. For example, the electrical potential of the conducting surface may vary between +5 volts and −5 volts compared with the electrical potential of the reference electrode. In other embodiments, the electrical potential of the conducting surface alternates between +3 volts and −3 volts compared with the electrical potential of the reference electrode. In yet other embodiments, the electrical potential of the conducting surface alternates by at least 3 volts and is maintained at a positive electrical potential compared with the electrical potential of the reference electrode. In other embodiments, the electrical potential of the conducting surface alternates is maintained between 0 volts and +10 volts compared with the electrical potential of the reference electrode. The electrical potential of the conducting surface may alternate, for example, at a frequency of between 0.1 and 10 hertz, or between 10 hertz and 100 hertz, or between 10 hertz and 20 hertz.

In one embodiment, the conducting surface is the surface of a medical device that is at least partially implanted within the body of a patient. For example, the medical device may be an osseointegrated prosthetic limb, a fracture fixation plate, or a total or partial joint replacement such as mandibular joint, a hip joint, a knee joint, an ankle joint or a shoulder joint. In one preferred embodiment, the medical device is an osseointegrated prosthetic limb.

The conducting surface of the medical device may include a material such as stainless steel, cobalt, chromium, molybdenum, titanium, vanadium, aluminum or a mixture or alloy of at least two of these materials. In one preferred embodiment, the conducting surface includes titanium.

The microbial growth may be bacterial growth, fungal growth or a combination thereof. For example, the bacterial growth may be staphylococcus growth. In one embodiment, the electrical current is varied to an extent sufficient to reduce or prevent biofilm formation on the conducting surface.

### BRIEF DESCRIPTION OF THE DRAWINGS

FIG. 1 is a schematic illustration of an apparatus for performing one embodiment of the present invention.

### DETAILED DESCRIPTION OF THE PREFERRED EMBODIMENTS

### Definitions

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention.

The uses of the terms “a” and “an” and “the” and similar references in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”) provided herein, is intended merely to better illuminate the invention and does not pose a limitation on the scope of the invention unless otherwise claimed. No language in the specification should be construed as indicating any non-claimed element as essential to the practice of the invention.

The term “therapeutic effect” as used herein means an effect which induces, ameliorates or otherwise causes an improvement in the pathological symptoms, disease progression or physiological conditions associated with or resistance to succumbing to a disorder, for example, a microbial infection in a human or veterinary patient.

As used herein, the term “patient” refers to a human or veterinary patient. In one embodiment, the veterinary patient is am mammalian patient.

A term “potentiostat” as used herein refers to an electronic instrument that controls the voltage difference between a working electrode and a reference electrode. The potentiostat implements this control by injecting current into the system through a counter electrode. There are a variety of potentiostats that are commercially available (e.g. Series G300™, Gamry Instruments Inc., Warminster, Pa., USA; SP-240, Biologic Science Instruments SAS, Claix, France).

### Method of Preventing or Reducing Microbial Growth on a Conducting Surface

One aspect of the invention provides a method of preventing or reducing microbial growth on a conducting surface. In one preferred embodiment, the conducting surface is a surface of a device that is at least partially implanted within the body of a patient. One preferred example of a partially implantable device is an osseointegrated prosthetic limb. In other embodiments, the device is a device that is totally implanted within the body of a patient, such as a fracture fixation plate, a pin, or an artificial joint. Examples of artificial joints include, but are not limited to, a hip joint, a knee joint, an ankle joint or a shoulder joint.

In other embodiments, the device is any device that includes a surface that is conducting or at least semi-conducting. For example, the surface may be a metallic surface, such as a surface including stainless steel, cobalt, chromium, molybdenum, titanium, vanadium, aluminum or a mixture or alloy of at least two of these materials.

In one embodiment, the surface of the device forms the working electrode of a three electrode potentiostat/galvanostat electrochemical cell. Such a cell also includes a counter electrode and a reference electrode, as well as a potentiostatic device. Preferably, the electrochemical cell is operated so that the electrical potential of the device surface (working electrode) is maintained at to positive electrical potential with respect to the electrical potential of the reference electrode by passing an electrical current through the working and counter electrodes.

The present method may include imparting an intermittent alteration of surface potential of the device surface to produce an electrochemically inhospitable environment for microbial/biofilm buildup. In certain embodiments, the potential of the device surface with respect to that of the reference electrode varied in a cyclic manner. For example the potential of the device surface may be varied at a frequency of between 0.1 and 10 Hz, 10 Hz and 200 Hz, or 10 Hz and 150 Hz, or 10 Hz and 100 Hz, or 10 Hz and 50 Hz.

In certain embodiments, the potential of the device surface varies within +10V and −10V relative to that of the reference electrode. In other embodiments, the potential varies between +5V and −5V, or +3V and −3V, or +2V and −2V or +1V and −1V. In other embodiments, the potential varies between 0 V, +0.1V, +0.5V, or +1.0V and +10V, or between +1V and +9V, or +2V and +8V, or +3V and +7V, or +4V and +6V, or +3V and +5V.

In yet other embodiments, the potential of the device surface varies by at least 3V, 4V 5V, 6V, 7V, 8V, 9V or 10V and is maintained at a positive potential with respect to the potential of the reference electrode. In other embodiments, the potential of the device surface varies by at least 3V, 4V 5V, 6V, 7V, 8V, 9V or 10V.

In one preferred embodiment, the potential at the device surface is varied to an extent sufficient to reduce or prevent microbial growth on the device surface and /or to prevent or reduce the formation of a biofilm on the device surface. In other embodiments, the potential at the device surface is varied for a time and to an extent sufficient to therapeutically affect the extent of microbial growth on the device surface and/or to provide a therapeutically affective treatment to the patient.

For example, the potential or cyclic variation in potential may be applied to the device surface for a period of between 1 min, and 5 min, 10 min, 20 min, 40 min or 1 hr. In other embodiments, the potential or variation in potential may be applied to the device surface for longer periods, for example, between 1 hr and 2 hr, 4 hr, 6 hr, 12 hr or 24 hr of for longer periods.

In one embodiment, the present method provides for the elimination or reduction of microbial growth on a conductive surface while minimizing untoward effects on the underlying tissue. The capability of inducing an alternating or positive electric current/potential whereby the microbial colony/biofilm on the surface is subject to irreversible membrane permeabilization processes, direct oxidation of cellular/viral constituents by electric current, and disinfection by electrochemically generated oxidants provides for the effective treatment of microbial infections on the implant surface.

The microbial growth may be, for example, a bacterial growth, a fungal growth and a combination thereof. The bacteria may be gram positive gram negative bacteria. For example, the bacteria may be a staphylococcus bacteria. In certain embodiments, the present method is used in combination with other treatment methods. Such treatment methods may include, but are not limited to, administrating an antimicrobial agent, such as an antibiotic, to the patient.

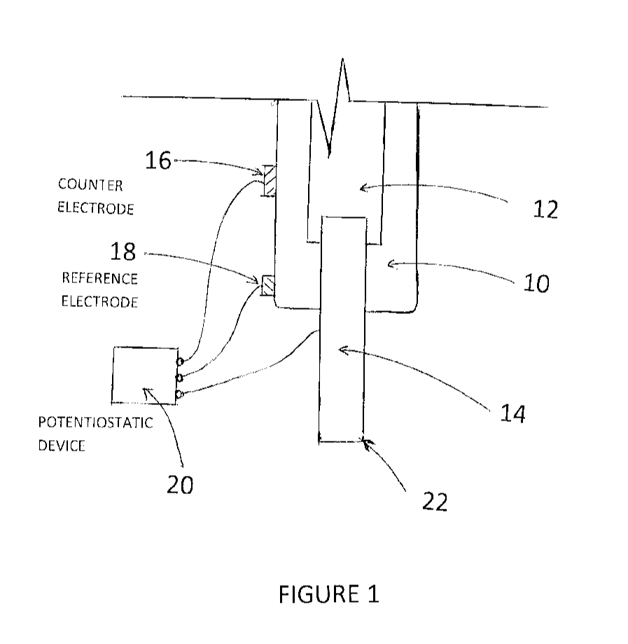

Turning to FIG. 1. This figure illustrates one embodiment of a method of the invention. Osseointegrated prosthetic device 14 is, for example, a prosthetic limb, and includes conductive surface 22. A portion of prosthetic device 14 is shown inserted into the intramedullary canal of the bone 12 of residual limb 10. Another portion of prosthetic device 14 extends from limb 10. Potentiostat 20 is electrically connected to counter electrode 16, which is illustrated in position on an external surface of residual limb 10. Reference electrode 18 is also in electrically connected to potentiostat 20 and is positioned on the external surface of limb residual 10. Finally, potentiostat 20 is electrically connected to an exposed portion of conducting surface 22 of prosthetic device 14.

Reference electrode 18, counter electrode 16 and conductive surface 22 of prosthetic device 14 form a three electrode electrochemical cell. Potentiostat 20, or a similar device providing a constant voltage or current (for example the PowerPack™ Basic Power Supply, BioRad, Hercules, Calif.) provides an electrical current through conducting surface 22 (the working electrode electrode) and counter electrode 16 to control the potential of conducting surface 22 with respect to the potential of reference electrode 18. As is disclosed above, in one embodiment, the potential of conducting surface is varied in a cyclic manner with respect to the potential of reference electrode 18. In another embodiment, the potential of conducting surface 22 is maintained at a positive potential with respect to reference electrode 18.

In one embodiment, the reference electrode includes silver or silver chloride. In another embodiment, the counter electrode includes platinum or graphite. The reference and counter electrodes may be, for example, pellet, wire or disc-type electrodes. In one embodiment, the counter and reference electrodes are attached to the skin of the patient.

In other embodiments, the conducting surface (working electrode) is a surface of a device that is completely implanted within the body of a patient. For example, the device may be an artificial joint. In such embodiments an electrically conductive material, such as a wire or needle, may be inserted into the body of the patient to contact the implant and connect the conductive surface to an external potentiostatic device. In other embodiments, the potentiostatic device and/or at least one of the reference and counter electrodes may also be implanted within the body of the patient.

Another aspect of the invention provides an apparatus for the treatment of microbial growth on a conducting surface. The apparatus may include a potentiostatic device in electrical contact with a reference electrode, a counter electrode and the conducting surface of the implantable device. In certain embodiments, one or more of these components is integrated into the implantable device. For example, the potentiostatic device may be at least partially contained within the implantable device. The reference and counter electrodes may also be included in the implantable device and may form separate portions of the device surface.

The power for an implanted potentiostatic device may be provided by an external device, or alternative may be an internal battery. In other embodiments, power may be supplied by a wireless methods, for example, an inductively charged power source.

### REFERENCES

1. Kang N V, Pendegrass C, Marks L, Blunn G. Osseocutaneous Integration of an Intraosseous Transcutaneous Amputation Prosthesis Implant Used for Reconstruction of a Transhumeral Amputee: Case Report. The Journal of Hand Surgery. 2010 July; 35(7):1130-4.

2. Hagberg K, Brånemark R. One hundred patients treated with osseointegrated transfemoral amputation prostheses—rehabilitation perspective. J Rehabil Res Dev. 2009; 46(3):331-44.

3. Waters R L, Perry J, Antonelli D, Hislop H. Energy cost of walking of amputees: the influence of level of amputation. J Bone Joint Surg Am. 1976; 58(1):42-6.

4. Sullivan J, Uden M, Robinson K P, Sooriakumaran S. Rehabilitation of the trans-femoral amputee with an osseointegrated prosthesis: The United Kingdom experience. Prosthetics and Orthotics International. 2003; 27(2):114-20.

5. Pendegrass C J, Goodship A E, Blunn G W. Development of a soft tissue seal around bone-anchored transcutaneous amputation prostheses. Biomaterials. 2006; 27(23):4183-91.

6. Tillander J, Hagberg K, Hagberg L, Brånemark R. Osseointegrated titanium implants for limb prostheses attachments: infectious complications. Clinical Orthopaedics and Related Research®. 2010; 468(10):2781-8.

7. Szkotak R, Niepa T H R, Jawrani N, Gilbert J L, Jones M B, Ren D. Differential Gene Expression to Investigate the Effects of Low-level Electrochemical Currents on Bacillus subtilis. AMB Express. 2011; 1(1):39.

8. Stein A, Bataille J F, Drancourt M, Curvale G, Argenson J N, Groulier P, et al. Ambulatory Treatment of Multidrug-Resistant-Staphylococcus-Infected Orthopedic Implants with High-Dose Oral Co-trimoxazole (Trimethoprim-Sulfamethoxazole). Antimicrobial agents and chemotherapy. 1998; 42(12):3086-91.

9. Stewart P S, William Costerton J. Antibiotic resistance of bacteria in biofilms. The Lancet. 2001; 358(9276):135-8.

10. lsraelachvili J N. Intermolecular and surface forces: revised third edition [Internet]. Academic press; 2011 [cited 2014 Jun. 23]. Available from:http://books.google.com/books?hl=en&lr=&id=vgyBJbtNOcoC&oi=fnd&pg=PP2&dq=Intermolecular+and+Surface+Forces&ots=_ONn9kA1Pa&sig=4Ld2a rnJOFMRuJpKV4NBxJUrB-A

11. Poortinga A T, Bos R, Busscher H J. Charge transfer during staphylococcal adhesion to TiNOX® coatings with different specific resistivity. Biophysical chemistry. 2001; 91(3):273-9.

12. Van der Borden A J, Maathuis P G, Engels E, Rakhorst G, van der Mei H C, Busscher H J, et al. Prevention of pin tract infection in external stainless steel fixator frames using electric current in a goat model. Biomaterials. 2007; 28(12):2122-6.

Although the invention has been described and illustrated with reference to specific illustrative embodiments thereof, it is not intended that the invention be limited to those illustrative embodiments. Those skilled in the art will recognize that variations and modifications can be made without departing from the true scope and spirit of the invention as defined by the claims that follow. It is therefore intended to include within the invention all such variations and modifications as fall within the scope of the appended claims and equivalents thereof.

We claim:

1. A method of treating microbial growth on a conducting surface, the method comprising:
applying a electrical current through the conducting surface and a counter electrode, wherein the electrical current is varied such that an electrical potential of the conducting surface alternates compared to the electrical potential of a reference electrode and wherein the electrical current is varied to an extent sufficient to reduce or prevent microbial growth on the conducting surface.

2. The method of claim 1, wherein the applying of the electrical current is performed by a potentiostat device.

3. The method of claim 1, wherein the electrical potential of the conducting surface alternates between +5 volts and −5 volts compared with the electrical potential of the reference electrode.

4. The method of claim 3, wherein the electrical potential of the conducting surface alternates between +3 volts and −3 volts compared with the electrical potential of the reference electrode.

5. The method of claim 1, wherein the electrical potential of the conducting surface alternates by at least 3 volts and is maintained at a positive potential compared with the electrical potential of the reference electrode.

6. The method of claim 1, wherein the electrical potential of the conducting surface alternates is maintained between 0 volts and +10 volts compared with the electrical potential of the reference electrode.

7. The method of claim 3, wherein the electrical potential of the conducting surface alternates at a frequency of between 10 hertz and 100 hertz.

8. The method of claim 7, wherein the electrical potential of the conducting surface alternates at a frequency of between 10 hertz and 20 hertz.

9. The method of claim 1, wherein the conducting surface is a surface of a medical device at least partially implanted within a patient.

10. The method of claim 9, wherein the medical device selected from the group consisting of an osseointegrated prosthetic limb, a fracture fixation plate, a hip joint, a knee joint, an ankle joint and a shoulder joint.

11. The method of claim 10, wherein the medical device is an osseointegrated prosthetic limb.

12. The method of claim 9, wherein the conducting surface comprises a material selected from the group consisting of stainless steel, cobalt, chromium, molybdenum, titanium, vanadium, aluminum and a mixture or alloy of at least two of these materials.

13. The method of claim 1, wherein the microbial growth is selected from the group consisting of a bacterial growth, a fungal growth and a combination thereof.

14. The method of claim 13, wherein the microbial growth is bacterial growth and wherein the bacterial growth is staphylococcus growth.

15. The method of claim 1, wherein the electrical current is varied to an extent sufficient to reduce or prevent biofilm formation on the conducting surface.

16. A method of treating microbial growth on a conducting surface, the method comprising:
applying a electrical current through the conducting surface and a counter electrode, wherein the conducting surface is a surface of a device at least partially implanted within a patient's body, wherein the electrical current is varied such that an electrical potential of the conducting surface alternates by at least 3 volts and is maintained at a positive potential compared with the electrical potential of the reference electrode and wherein the electrical current is varied to an extent sufficient to reduce or prevent microbial growth on the conducting surface.

17. The method of claim 16, wherein the electrical potential of the conducting surface alternates at a frequency of between 10 hertz and 100 hertz.

18. The method of claim 16, wherein the medical device selected from the group consisting of an osseointegrated prosthetic limb, a fracture fixation plate, a hip joint, a knee joint, an ankle joint and a shoulder joint.
